(19) United States
(12) Patent Application Publication
(10) Pub. No.: US 2005/0084553 A1
(43) Pub. Date: Apr. 21, 2005

(54) Composition containing moutan root bark extract as active ingredient

(76) Inventors: Woong-Sig Moon, Gyeonggi-do (KR); Jae-Chul Kim, Gyeonggi-do (KR); Heun-Soo Kang, Seoul (KR); Seong-Min Sohn, Seoul (KR)

Correspondence Address: ALSTON & BIRD LLP;BANK OF AMERICA PLAZA, 101 SOUTH TRYON STREET, SUITE 4000, CHARLOTTE, NC, 28280-4000, US

(21) Appl. No.: 10/502,520

(22) PCT Filed: Jan. 24, 2003
(86) PCT No.: PCT/KR03/00160

(30) Foreign Application Priority Data
Jan. 26, 2002 (KR) 2002-4660
Jul. 30, 2002 (KR) 2002-45033
Jan. 24, 2003 (KR) 2003-4683

(51) Int. Cl. A61K 35/78; A61K 31/496; A61K 31/4196; A61K 31/4178; A61K 31/137

(52) U.S. Cl. 424/773; 514/254.07; 514/27; 514/383; 514/397; 514/649

(57) ABSTRACT

The present invention relates to a composition comprising a Moutan root bark extract as an active ingredient, and particularly to a composition for the prevention and treatment of athlete's foot, osmidrosis, or acne; a composition for oral cleansing; a bactericidal/disinfectant composition; a composition for eliminating dandruff-causing pathogens and controlling odor, and a functional kitchen detergent composition. The compositions comprising the Moutan root bark extract as an active ingredient of the present invention have superior bactericidal effects against bacteria including athlete's foot-causing pathogens, skin eczema-causing pathogens and skin flora, and fungi, and they can be easily and simply applied to medicines, cosmetics, or cleansing agents.

### BACKGROUND OF THE INVENTION

(a) Field of the Invention

The present invention relates to a composition comprising a Moutan root bark extract as an active ingredient, and more particularly, to a composition for the prevention and treatment of athlete's foot, osmidrosis, or acne, a composition for oral cleansing, a bactericidal/disinfectant composition, and a kitchen detergent composition applicable to medicines, cosmetics or cleansing agents.

(b) Description of the Related Art

Bacteria or fungi exist throughout the world, and there are many various kinds. Bacteria and fungi grow and reproduce under suitable growth conditions, and they may also induce diseases in animals including humans. In particular, candidiasis and athlete's foot due to fungi frequently occur, and microbes cause damage to the quality and performance of foods or industrial products.

Fungi are parasitized in skin and give rise to dermato-mycosis. In particular, dermatophytes parasitized in keratin tissues such as the keratin of skin, hair, fingernails, and toenails may also cause dermato-phytosis, Tinea, or superficial fungal infection. The main source of the superficial fungal infection is microsporum, epidermo-phyton, Trichophyton, candida species, and Malassezia furfur. The bacteria causing the superficial fungal infection mostly induce superficial lesions by proliferating in the keratin tissues of upper epithelial cells, but sometimes they may induce inflammation below the upper part of epithelia and also give rise to Dermatophytid.

As a disease generated by bacteria, Osmidrosis can be mentioned. Osmidrosis is a disease generating a foul odor caused by microbes residing in the axilla, by which substances of the skin surface including apocrinal gland secretion, keratin secretion, sebum, and sweat are degraded.

The bacteria causing osmidrosis include aerobic diptheroid, coaglucoccus negative Staphylococci, etc. (Korean Journal of Dermatology, Vol. 28, No. 5, pp. 559-564, 1990). In order to resolve such osmidrosis, methods of inhibiting the secretion of sweat or inhibiting the degradation of sweat have been attempted, and research on using direct or indirect microbe inhibitors or enzyme inhibitors such as ethyl lactate, octylcrotonate, triethyl citrate, carathane(4,6-dinitro-2-(methylheptyl)phenylcrotonate), etc. has been conducted. In addition, given that the oxidative substances of a large amount of unsaturated compounds contained in sebum contribute to the generation of foul odor, anti-oxidant agents such as BHA (Butylate HydroxyAnizol) or BHT (Butylated HydroxyToluene) have been used. However, such methods showed poor deodorization effects, and they may give rise to serious damage to skin. The use of the inhibitors of sweat secretion may impede normal homostasis, and microbe inhibitors and anti-oxidant agents may cause skin toxicity, and in many cases they can induce skin stimulation by lowering its pH.

Bacteria are divided into useful bacteria that are beneficial to humans, and noxious bacteria that cause damage. The useful bacteria are used in food processing or as antibiotics. As typical noxious bacteria that give rise to diseases, there are clostridium tetati, comma bacillus, C. diphtheriae, and tubercle bacillus. Particularly, pathogenic bacteria cause fever or inflammation reaction by infecting animals including humans.

In prior arts, in order to suppress the activities of the noxious microbes, synthetic organic antibacterial agents or inorganic antibacterial agents have been used. However, the existent synthetic, organic antimicrobial agents have the drawbacks of causing strong stimulation to eyes, skin, or olfactory sense, and of exhibiting weak antibacterial activities against gram-negative bacteria (presence of cellular membrane). Also, the inorganic antibacterial agents generally exhibit weak antibacterial abilities, and in particular, they exhibit little antibacterial activity against fungi and their antibacterial abilities are suddenly reduced when they come into contact with moisture.

As typical natural antibacterial agents, which are currently available, there are polylysines or niacins which are the bacteriocins of Streptomyces sp.

However, a large amount of them is required for antibacterial activities, and they are very expensive. Further, chito acids are more effective against gram-positive bacteria than against gram-negative bacteria, but they show relatively weak antibacterial abilities against fungi, and furthermore, their effects are insignificant against genuses containing chito acids within their cell walls (Riccardo M et al., Antimicrobial Society and Chemotherapy, 1990, 34, 2019-2023).

To prevent and treat infection of bacteria or fungi, Korea Laid-Open Patent No. 90-17490 discloses a composition having dosage forms such as powders, solutions, suspensions, creams, gels, pastes, ointments, or tinctures as a pharmaceutical composition suitable to the treatment of fungal skin diseases by local administration, and a cosmetic composition. However, in the case that the composition of such dosage forms are applied to the affected parts, a large quantity of medicine is required when the parts to be applied are broad, controlling the amount to be applied uniformly is difficult, and semi-solids with high viscosity (ex.: ointments and creams) have a problem of adhering to clothes after being applied to skin.

Further, in the case of antibacterial or antifungal agents for oral administration, the medicines are circulated in the whole body, and thus in order to have an effect from the medicines, an excessive dose must be administered, and side effects due to long-term medication may be generated. Particularly, toxicity in the body from triazole medicines has become problematic: for example, they give rise to side effects in the liver when taken for a long-term period.

### SUMMARY OF THE INVENTION

The present invention has been made to solve the problems the prior arts as mentioned above, and it is an object of the invention to provide a composition that is harmless to a human body and has excellent antibacterial activity against dermatophytes such as athlete's-foot-causing pathogens and dandruff-causing pathogens.

It is another object of the invention to provide a composition for the prevention of osmidrosis and for the treatment of Osmidrosis.

It is a further object of the invention to provide a composition for the prevention and treatment of acne.

It is a still further object of the invention to provide an antimicrobial spray composition that can be easily and simply applied to affected parts, and that can enable antibacterial and antifungal substances to readily permeate into skin.

Further, it is an object of the invention to provide an antimicrobial spray composition that can prevent and treat diseases due to bacteria or fungi and that is harmless to human body.

Still further, it is an object of the invention to provide a composition capable of effectively sterilizing and/or disinfecting noxious microbes distributed in nature.

Still further, it is an object of the invention to provide a composition for the inhibition of halitosis and the prevention of tooth decay having an antibacterial activity against noxious oral microbes.

Further, it is an object of the invention to provide a kitchen detergent composition capable of effectively inhibiting noxious microbes that can be seen in ordinary life.

In order to achieve the aforementioned objects, the present invention provides a composition for the prevention and treatment of athlete's foot comprising a Moutan root bark extract as an active ingredient.

Also, the invention provides a composition for the prevention of osmidrosis comprising a Moutan root bark extract as an active ingredient.

Also, the invention provides an anti-acne composition comprising a Moutan root bark extract as an active ingredient.

Also, the invention provides an antimicrobial composition comprising a Moutan root bark extract, and one or more compounds selected from the group consisting of Ketoconazole, Itraconazole, Fluconazole, Miconazole, Clotrimazole, Fenticonazole, Econazole, Bifonazole, Oxiconazole, Cloconazole, Rolcyclate, Amphotericin B, Flucytosine, Griceofulvin, Terbinafine, Nystatin, Tolnaftate, Naftifine, Haloprogin, Ciclopirox, and Triclosan.

Also, the invention provides a bactericidal disinfectant composition comprising a Moutan root bark extract, and one or more compounds selected from the group consisting of norfloxacin, ciprofloxacin, ciprofloxacin salt, itraconazole nitrate, mitoconazole nitrate, and ketoconazole.

Also, the invention provides a composition for oral cleansing comprising a Moutan root bark extract, and one or more compounds selected from the group consisting of xylitol, propolis, triclosan, chlorohexidine gluconate (XII), cetyl pyridinium chloride (XIII), isopropylmethylphenol, hitokitiol, glytylitylic acid, and allantoin.

Also, the invention provide a kitchen detergent composition comprising a Moutan root bark extract, and one or more compounds selected from the group consisting of Ketoconazole, Itraconazole, Fluconazole, Miconazole, Clotrimazole, Fenticonazole, Econazole, Bifonazole, Oxiconazole, Cloconazole, Rolcyclate, Amphotericin B, Flucytosine, Griceofulvin, Terbinafine, Nystatin, Tolnaftate, Naftifine, Haloprogin, Ciclopirox, and Triclosan.

### DETAILED DESCRIPTION OF THE PREFERRED EMBODIMENTS

The inventors found that the Moutan root bark extract effectively functions against athlete's foot, osmidrosis, halitosis, tooth decay, and acne, and thereby completed the subject invention.

The Moutan root bark extract of the invention is prepared by extracting Moutan root bark according to conventional extraction methods.

Moutan root bark, the root bark of Paeonia Suffruticosa Andrews, is a substance that has been used medicinally in oriental medicine, and it is harmless to a human body.

Moutan root bark extract, for example, can be prepared by dipping dried plant roots into an extraction solution to prepare an extract, and concentrating it under a reduced pressure, or it can be obtained by separating layers with an extraction solution. The extraction solution may be one or more compounds selected from the group consisting of water, ethyl acetate, butyl acetate, ethyl alcohol, isopropyl alcohol, butyl alcohol, hexane, chloroform, ethyleneglycol, propyleneglycol, propanol, acetone, benzene, ethanol, methanol, and butanol.

The present invention provides a composition for the prevention and treatment of athlete's foot comprising a Moutan root bark extract as an active ingredient.

The composition for the prevention and treatment of athlete's foot of the invention may comprise the Moutan root bark extract alone, or it may comprise 0.001 to 40% by weight of Moutan root bark extract and the remaining amount as pharmacologically acceptable substances.

Further, the composition for the prevention and treatment of athlete's foot of the invention may further comprise one or more compounds selected from the group consisting of Ketoconazole, Itraconazole, Fluconazole, Miconazole, Clotrimazole, Fenticonazole, Econazole, Bifonazole, Oxiconazole, Cloconazole, Rolcyclate, Amphotericin B, Flucytosine, Griceofulvin, Terbinafine, Nystatin, Tolnaftate, Naftifine, Haloprogin, Triclosan, and Ciclopirox, and the content of these compounds may be 0.001 to 30% by weight with regard to the total composition. If the content of such compounds is less than 0.001% by weight, the antibacterial effect may be insignificant, and if it exceeds 30% by weight, the compounds may be precipitated or the economic efficiency may be low in comparison with the effect obtained. The preferred content of the compounds is 0.05 to 10% by weight.

The composition for the prevention and treatment of athlete's foot of the invention can be manufactured in an oral or parenteral form, and its dosage form may be plasters, granules, lotions, liniments, lemonades, aromatic waters, powders, syrups, liquids and solutions, aerosols, sprays, extracts, elixirs, ointments, fluidextracts, emulsions, suspensions, decoctions, infusions, tablets, injections, capsules, creams, tinctures, pastes, or pills. Preferably, the composition for the prevention and treatment of athlete's foot is manufactured in the form of aerosols or sprays.

The composition for the prevention of athlete's foot of the present invention may be medicines, cleansing agents, or cosmetics. In the case of medicines, they may be directly applied to the affected parts, and the cleansing agents may be soaps. The cosmetics may be in the form of lotions or massage creams.

Also, the present invention provides a composition for the prevention of osmidrosis comprising a Moutan root bark extract as an active ingredient. The composition for the prevention of osmidrosis of the invention may comprise the Moutan root bark extract alone, or it may comprise 0.001 to 40% by weight of Moutan root bark extract and the remaining amount as pharmacologically acceptable substances. As said substances that may be further included, there are Triclosan and aluminium hydroxychloride.

The composition for the prevention of osmidrosis of the invention can be manufactured in a parenteral form, and its dosage form may be lotions, powders, syrups, liquids and solutions, sticks, gels, aerosols, sprays, ointments, emulsions, suspensions, or creams. The preferred dosage forms are aerosols or sprays.

Also, the present invention provides an anti-acne composition comprising a Moutan root bark extract as an active ingredient. The anti-acne composition of the invention may comprise the Moutan root bark extract alone, or it may comprise 0.001 to 40% by weight of Moutan root bark extract and the remaining amount as pharmacologically acceptable substances. As such substances, there are Triclosan or aluminium hydroxychloride.

The anti-acne composition of the invention can be manufactured in a parenteral form, and its dosage form may be lotions, powders, syrups, liquids and solutions, aerosols, sprays, ointments, emulsions, suspensions, or creams. The anti-acne composition of the invention can be applied to cosmetics, cleansing agents, or medicines, and the cosmetics or cleansing agents may be all kinds of forms applicable to human skin. For example, the cosmetics may be lotions, creams, emulsion solutions, gels, or packs, and the cleansing agents may be soaps.

The present invention also provides an anti-microbial composition comprising a Moutan root bark extract and anti-microbial compounds. The anti-microbial activity as referred to herein means a bactericidal activity against noxious microbes such as algae, bacteria, protozoa, mold, yeasts, or viruses, and as noxious microbes, there are germs causing athlete's foot, dermatomycosis-causing pathogens, skin flora, noxious bacteria, and superficial fungal infection-causing bacteria.

The anti-microbial compounds may be one or more compounds selected from the group consisting of Ketoconazole, Itraconazole, Fluconazole, Miconazole, Clotrimazole, Fenticonazole, Econazole, Bifonazole, Oxiconazole, Cloconazole, Rolcyclate, Amphotericin B, Flucytosine, Griceofulvin, Terbinafine, Nystatin, Tolnaftate, Naftifine, Haloprogin, and Ciclopirox. The preferred compound is Ketoconazole.

The anti-microbial composition of the invention may comprise the Moutan root bark extract and the anti-microbial compounds in a ratio of 1:5 to 5:1 by weight, and may further comprise other pharmacologically acceptable substances. In the case that the anti-microbial composition comprises substances other than the Moutan root bark extract and antimicrobial compounds, the Moutan root bark extract can be contained in the anti-microbial composition in an amount of 0.001 to 20% by weight and the anti-microbial compounds can be contained in the anti-microbial composition in an amount of 0.001 to 20% by weight. If the content of the Moutan root bark extract and the anti-microbial compounds is less than 0.001% by weight, anti-microbial activity may be insignificant, and if it exceeds 20% by weight, the economic efficiency may be low as their activity does not increase in proportion to the amount added. The preferred content of each of the Moutan root bark extract and the anti-microbial compounds is 1 to 10% by weight.

Further, the anti-microbial composition of the invention may further comprise conventional pharmacologically acceptable substances, in addition to the Moutan root bark extract and the anti-microbial compounds. As such substances, there are skin moisturizers, skin permeation enhancers, fragrances, fragrance encapsulation carriers, organic solvents, or fillers.

The skin moisturizers can be one or more compounds selected from the group consisting of ethylene glycol, propylene glycol, butylene glycol, hexylene glycol, polyethylene glycol (PEG) 200 to 600, polypropylene glycol (PPG), glycol ester and ether, alkyl ester of PEG or PPG, carboxylic ester of PEG or PPG, sorbitol, trihydroxy stearine, and polyhydric alcohol derivatives. The content of the skin moisturizers in the anti-microbial composition is preferably 0.05 to 5% by weight. If the content of the skin moisturizers is less than 0.05% by weight, sufficient moisturizing effects are not obtained, and if it, exceeds 5% by weight, the properties of the anti-microbial composition may be changed. More preferably, the content of the skin moisturizers is 0.1 to 3% by weight.

The skin permeation enhancers are used to facilitate the penetration into skin keratin and permeation into corium of the anti-microbial substances, and examples thereof include polyethylene glycol monolaurate (PEGML), glycerol monolaurate, propylene glycol monolaurate, eucalyptol, lecithin, 1-substituted azacycloheptane-2-one, 1-n-dodecyl cycleazacyclohepta-2-one (Trademark: IZON), cetyl alcohol, stearyl alcohol, myrist alcohol, polyethylene sorbitan fatty acid ester (ex.: Tween 20, 40, 60, 80, etc.), sorbitan fatty acid ester (ex: SPAN 60, 80, etc.), dodecyl amine or lanolin.

The skin permeation enhancers can be used alone or in a mixture of two or more kinds, and they are preferably selected from the group consisting of a Tween compound, which is a polyethylene sorbitan fatty acid ester; a SPAN compound, which is a sorbitan fatty acid ester; lanolin; and a mixture thereof. More preferably, lanolin is used. The content of the skin permeation enhancers in the anti-microbial composition is preferably 0.1 to 10% by weight. If the content of the skin permeation enhancers is less than 0.1% by weight, skin permeating effects may not be effectively attained, and if it exceeds 10% by weight, economic efficiency is low in comparison with the effects to be obtained. More preferably, the amount of the skin permeation enhancers is 0.5 to 5% by weight.

The fragrances are used for masking purposes to eliminate odor, and all kinds of fragrance that are added to conventional cosmetics or medicines and are applicable to humans or animals can be used. Examples of the fragrances include lavender, lemon, floral, herb, apple, strawberry, lily, frisia, lilac, rose, acacia, Chinese quince, musk, pheromones, and pine flavor, and they can be used alone or in a mixture of two or more kinds. It is preferred that the content of the fragrances in the anti-microbial composition is 0.05 to 2% by weight. If the content of the fragrances is less than 0.05% by weight, the masking effects may be insignificant, and if it exceeds 2% by weight, the strong odor may cause reverse effects. More preferably, the content of the fragrances is 0.5 to 1.5% by weight.

Fragrance encapsulation carriers are used to maintain odor-masking functions. The carriers can be conventional substances, and they are preferably dextrines or cyclodextrines. The content of the carriers in the anti-microbial composition is preferably 0.1 to 10% by weight, and more preferably 1.0 to 5% by weight. If the content of the carriers is less than 0.1% by weight, the encapsulating effects of the fragrances may be insignificant, and if it exceeds 10% by weight, this may be uneconomical.

The solvents are used to effectively dissolve or disperse each substance contained in the anti-microbial composition, and any solvents that are harmless to animals can be used. Preferred solvents include ethanol, isopropanol, or mixture thereof, and they can be contained in the spray composition in the remaining amounts.

As the fillers, any filler that are used in spray products can be used. As the typical fillers, liquefied petroleum gas (LPG) can be mentioned. Also, the fillers can be contained in a sufficient amount so that the anti-microbial composition can be applied in the form of sprays. The preferred content is 0.01 to 50% by weight.

The anti-microbial composition of the invention preferably has a pH of 3.0 to 9.0, and more preferably a pH of 4.0 to 7.5. If the pH is less than 3.0 or higher than 9.0, side effects with respect to the stability of the dosage form or application may occur.

The anti-microbial composition of the invention can be manufactured in the form of liquids, gels, solids, aerosols, or sprays, and according to the dosage form, conventional substances known to a skilled person in the pertinent art can be added.

In the case that the anti-microbial composition of the invention is manufactured in the form of sprays, the composition can be manufactured in the form of powder sprays or liquid sprays, and preferably liquid sprays are used. The spray composition can be used by spraying it onto either the skin of animals including humans, things that contact the skin, or the living environment of the animals, that is, it can be used by spraying it directly onto feet or on foul smelling shoes or socks. The anti-microbial spray composition can be filled into conventional spray containers such as plastic, aluminum, or tin containers with spray apparatuses.

The spray composition of the invention can be evenly applied to the affected parts in a constant amount, and it does not adhere to clothing, and thus its usability is superior and its hygienic and antibacterial and antifungal effects are excellent. In particular, the antimicrobial spray composition is effective on foot-and-mouth disease, skin itching, Tinea, dandruff, or athlete's foot.

The antimicrobial composition of the invention may be a detergent composition, a composition for controlling dandruff-causing pathogens, and odor, a bactericidal disinfectant composition, a composition for the sterilization and disinfection of animals and animal breeding farms, or an anti-foot-and-mouth disease composition.

The present invention also provides a composition for oral cleansing comprising a Moutan root bark extract and one or more compounds selected from the group consisting of xylitol, propolis, triclosan, chlorohexidine gluconate (XII), cetyl pyridinium chloride (XIII), isopropylmethylphenol, hitokitiol, glytylitylic acid, and allantoin.

The composition for oral cleansing of the invention may comprise the Moutan root bark extract and the above-mentioned compounds in a ratio of 1:5 to 5:1 by weight, and it my further comprise other pharmacologically acceptable substances. In the case that the composition for oral cleansing further comprises substances other than the Moutan root bark extract and antimicrobial compounds, the Moutan root bark extract can be contained in the composition for oral cleansing in an amount of 0.001 to 20% by weight and the antimicrobial compounds can be contained in the composition for oral cleansing in an amount of 0.001 to 20% by weight. If the respective contents of the Moutan root bark extract and the antimicrobial compounds are less than 0.001% by weight, the antimicrobial activity may be insignificant, and if they exceed 20% by weight, economic efficiency may be low as their activity does not increase in proportion to the amounts added. The preferred content of each of the Moutan root bark and the antimicrobial compounds is 0.1 to 10% by weight.

The composition for oral cleansing of the invention may further comprise substances that are used in conventional toothpaste compositions, gargling compositions, or compositions for the inhibition of halitosis and the prevention and treatment of decayed teeth, or otherwise the composition for oral cleansing can be used as an additive and be contained in toothpastes, mouthwashes, or agents for the inhibition of halitosis and the prevention of decayed teeth.

The composition for oral cleansing of the invention may be the dosage forms of plasters, granules, powders, syrups, liquids and solutions, aerosols, sprays, ointments, fluidextracts, emulsions, suspensions, tablets, capsules, creams, or pills.

Also, the present invention provides a kitchen detergent composition comprising a Moutan root bark extract and one or more compounds selected from the group consisting of Ketoconazole, Itraconazole, Fluconazole, Miconazole, Clotrimazole, Fenticonazole, Econazole, Bifonazole, Oxiconazole, Cloconazole, Rolcyclate, Amphotericin B, Flucytosine, Griceofulvin, Terbinafine, Nystatin, Tolnaftate, Naftifine, Haloprogin, Ciclopirox, Triclosan, norfloxacin, cifloxacin, and salts thereof.

The kitchen detergent of the invention may comprise the Moutan root bark extract and the above-mentioned compounds in a ratio of 1:5 to 5:1 by weight, and it may further comprise other pharmacologically acceptable substances. In the case that the kitchen detergent composition further comprises substances other than the Moutan root bark extract and antimicrobial compounds, the Moutan root bark extract can be contained in the kitchen detergent composition in an amount of 0.001 to 20% by weight and the antimicrobial compounds can be contained in kitchen detergent composition in an amount of 0.001 to 20% by weight. If the respective contents of the Moutan root bark extract and the antimicrobial compounds are less than 0.001% by weight, the antimicrobial activity may be insignificant, and if they exceed 20% by weight, economic efficiency may be low as their activity does not increase in proportion to the amounts added. The preferred content of each of the Moutan root bark and the antimicrobial compounds is 0.1 to 10% by weight.

The kitchen detergent composition of the present invention can comprise conventional compositions for the detergent of dishes and cooking utensils.

The dosage form of the kitchen detergent composition of the invention may be liquids and solutions, aerosols, or sprays.

Hereafter, examples of the present invention will be described. The following examples are provided solely to illustrate the subject invention: the subject invention should not be construed to be limited thereto.

### EXAMPLES 1 to 11

### Preparation of Plant Extracts

Moutan root bark, the root bark of Paeoniaceae (Paeonia suffruticosa Andrews), which is sold for medical purpose, was purchased. The Moutan root bark was completely dried and crushed, and then passed through 1.18-mm mesh. 10 kg of each solvent of Table 1 below were individually added to 1 kg of the crushed Moutan root bark, which was then placed at a room temperature for 8 hours. Thereafter, it was extracted at 45° C. for 12 hours, and then filtered with a Wattman #6 filter to yield a filtrate. The filtrate was concentrated in a distillation apparatus with a cooling condenser under a reduced pressure at 60° C. to yield extracts.

The yield of Moutan root bark extracts according to each solvent is exhibited in Table 1 below.

TABLE 1

| | Solvent | Moutan Root Bark Extract (g) |
|---|---|---|
| EX. 1 | water | 32 |
| EX. 2 | ethyl acetate | 17 |
| EX. 3 | butyl acetate | 25 |
| EX. 4 | ethyl alcohol | 33 |
| EX. 5 | isopropyl alcohol | 23 |
| EX. 6 | butyl alcohol | 17 |
| EX. 7 | ethylene glycol | 21 |
| EX. 8 | propylene glycol | 26 |
| EX. 9 | propanol | 31 |
| EX. 10 | acetone | 25 |
| EX. 11 | benzene | 31 |

### Experiment 1

1. Patch Test

The skin stimulation of the Moutan root bark extracts was investigated by patch tests.

Each plant extract was applied to a chamber (stiff aluminum) with a diameter of 8 mm and a depth of 0.5 mm, and five chambers were adhered to each of 2 rectangular tape lines. The tapes were adhered to 100 adults for two days, then the patches were removed and skin stimulation was evaluated according to ICDRG standards. As a result, there was no skin stimulation as shown in Table 2 below.

ICDRG Standards

?: doubtful reaction, +: weak positive, ++: strong positive, +++: ultra positive, −: negative, IR: stimulated

TABLE 2

| Extraction Solvent | Skin Stimulation |
|---|---|
| water | — |
| ethyl acetate | — |
| butyl acetate | — |
| ethyl alcohol | — |
| isopropyl alcohol | — |
| butyl alcohol | — |
| ethylene glycol | — |
| propylene glycol | — |
| propanol | — |
| acetone | — |
| benzene | — |

2. Verification of Antimicrobial Activity

As bacteria, E. coli 0-157:H7 (KCTC1682), Staphylococcus aureus (KCTC1621), Salmonella typhimutium (KCTC1925), and Listeria monocytogenes (ATCC 19111) were used; and as fungi, Candida albicans (KCTC7729), T. mentagrophytes (KCTC6085), T. rubrum (KCTC6352), A. niger (KCTC6985) P. citrinum (KCTC6990), and A. flavans (KCTC 6081) were used.

Antifungal activities were tested according to ASTM G21, which is the standard of the American Society for Testing and Material, and antibacterial activities were tested by the shake flask method. To determine the antibacterial activity, each strain was cultured while shaking at 25° C. for 24 hours (shaking rate: 150 times/min.), and then the number of strains was measured by harvesting a portion of the culture solutions from the experimental groups that were cultured by addition of 20 μl of Moutan root bark extract and the control groups that were cultured with no addition. Antibacterial activity was converted according to the following equation 1, and the results are exhibited in Table 3 below.
Antibacterial Activity=((Number of Strains in Control Group−Number of Strains in Experimental Group)/Number of Strain in Control Group)×100 (Equation 1)

Antifungal activity was tested according to ASTM G21, which is the standard of the American Society for Testing and Material. The grade of antifungal activity was determined by the following criteria, and the results are exhibited in Table 3 below.

Criteria

grade 0: No strain was grown on specimen

grade 1: Strain within 10% was grown on specimen

grade 2: Strains of about 10%˜30% were grown on specimen

grade 3: Strains of about 30%˜60% were grown on specimen

grade 4: Strains more than 60% were grown on specimen

TABLE 3

| Strain | Antimicrobial Activity |
|---|---|
| E. coli 0-157: H7 | 99.99 |
| S. aureus | 99.99 |
| S. chlerasuis | 100 |
| P. hacmolytica | 100 |
| L. monocytogenes | 100 |
| S. typhimurium | 99.99 |
| T. mentagrophytes | grade 0 |
| T. rubrum | grade 0 |
| Candida albicans | grade 0 |
| A. niger | grade 0 |
| P. citrinum | grade 0 |
| A. flavans | grade 0 |

From Table 3 above, it can be seen that the Moutan root bark extracts of the present invention were excellent in antibacterial and antifungal activities.

### EXAMPLES 12 to 16

### Preparation of Liquid Bactericidal, Disinfectant Agent

The bactericidal disinfectant compositions containing the Moutan root bark of Examples 1 to 11 and natural herb substances to confer an odor masking function, with which purified water and ethanol were mixed, were prepared as shown in Table 4 below (units: wt. %).

The determination of bactericidal ability was conducted according to the strain reduction rate determination method (Shaking flask method), by observing bacteria reduction rate after 24 hours from the time E. coli (KCTC1682), Staphylococcus aureus (KCTC 1621), and Salmonella typhimurium (KCTC1925), which are readily found in daily life, were inoculated, and the results are as shown in Table 4 below.

TABLE 4

| Compositional Ingredient | | EX. 12 | EX. 13 | EX. 14 | EX. 15 | EX. 16 |
|---|---|---|---|---|---|---|
| Moutan Root Bark Extract | | 10 | 10 | 5 | 10 | 10 |
| Lavender | | — | 0.5 | 0.5 | — | — |
| Rosemary | | — | — | — | 0.5 | — |
| Peppermint | | — | — | — | — | 0.5 |
| Ethanol | | 80 | 19.5 | 15 | 19.5 | 19.5 |
| Purified Water | | 10 | 70 | 79.5 | 70 | 70 |
| Total | | 100 | 100 | 100 | 100 | 100 |
| Strain | E. coli | 99.99 | 99.36 | 87.01 | 99.50 | 99.99 |
| Reduction | S. aureus | 99.99 | 96.95 | 86.33 | 99.99 | 99.97 |
| Rate (%) | S. typhimurium | 99.99 | 97.43 | 86.14 | 99.43 | 99.20 |

From Table 4, it can be seen that the Moutan root bark extracts of the invention still maintained excellent antibacterial activities even when they were liquids and solutions comprising the fragrances.

### EXAMPLES 17 to 21

With the compositional ratios of Table 5 below, antimicrobial agents were prepared and their antibacterial activities were verified.

TABLE 5

| | | EX. 17 | EX. 18 | EX. 19 | EX. 20 | EX. 21 |
|---|---|---|---|---|---|---|
| Moutan Root Bark Extract | | 10 | 10 | 10 | 5 | 5 |
| Ciprofloxacin | | — | 5 | — | — | — |
| Ciprofloxacin Salt | | 1 | — | — | 1 | 1 |
| Enosaxin | | — | — | 2 | — | — |
| Norfloxacin | | — | — | — | 1 | 1 |
| Itraconazole Nitrate | | — | — | 2 | — | — |
| Mitoconazole Nitrate | | — | 5 | — | 1 | — |
| Ketoconazole | | 3 | — | — | 1 | 2 |
| Triclosan | | 1 | — | 1 | 1 | 1 |
| Ethanol | | 25 | 20 | 25 | 30 | 30 |
| Purified Water | | 60 | 60 | 60 | 60 | 60 |
| Total | | 100 | 100 | 100 | 100 | 100 |
| Antibacterial | E. coli | 99.99 | 99.99 | 99.99 | 99.99 | 99.99 |
| Activity (%) | S. aureus | 99.99 | 99.99 | 99.99 | 99.99 | 99.99 |
| | S. typhimurium | 99.99 | 99.99 | 99.99 | 99.99 | 99.99 |
| Antifungal | P. citrium | grade 0 | grade 0 | grade 0 | grade 0 | grade 0 |
| Activity | A. niger | grade 0 | grade 0 | grade 0 | grade 0 | grade 0 |

From Table 5 above, it can be seen that the antimicrobial agents comprising the Moutan root bark ethyl acetate extract and antimicrobial compounds had excellent antibacterial and antifungal activities.

### EXAMPLES 22 TO 25

With the compositional ratios of Table 6 below (units: wt. %), the bactericidal disinfectant agents were prepared by mixing the Moutan root bark ethyl acetate extract, antimicrobial compounds, fragrances, and solvents. Thereafter, antibacterial, antifungal and anti-foot-and-mouth disease activities were tested.

Anti-foot-and-mouth disease activity was measured by the following method.

1. Antimicrobial agent and foot-and-mouth disease virus were contacted at 4° C. for 30 minutes.

2. BHK21 (cell for investigating the presence of virus) was inoculated thereto and then cultured at 37° C. for 1 hour.

3. The culture was inoculated on agar media and then incubated at 37° C. for 48 hours.

4. After dyeing it with methylene blue, whether or not plaque was formed was observed under the presence of natural light.

TABLE 6

| | | EX. 22 | EX. 23 | EX. 24 | EX. 25 |
|---|---|---|---|---|---|
| Moutan Root Bark | | 5 | 5 | 5 | 5 |
| Norfloxacin | | — | — | — | — |
| Ciprofloxacin | | 1.0 | — | 1.0 | — |
| Ciprofloxacin Salt | | — | 1.0 | — | 1.0 |
| Rosemary | | — | — | — | 0.5 |
| Acyclovir | | 1.0 | 1.0 | 1.0 | 1.0 |
| Ribavirin | | 1.0 | 1.0 | 1.0 | 1.0 |
| Triclosan | | 1.0 | — | — | 2.0 |
| Lavender | | 0.5 | — | — | — |
| Sodium Hydrogen carbonate | | 1.0 | — | 1.0 | — |
| Ethanol | | 15.0 | 15.0 | 15.0 | 15.0 |
| Purified Water | | 74.5 | 77.0 | 76.0 | 74.5 |
| Total | | 100 | 100 | 100 | 100 |
| Antibacterial | E. coli | 99.99 | 99.99 | 99.99 | 99.99 |
| Activity (%) | S. aureus | 99.99 | 99.99 | 99.99 | 99.99 |
| | S. typhimurium | 99.99 | 99.99 | 99.99 | 99.99 |
| | L. monocytogenes | 99.99 | 99.99 | 99.99 | 99.99 |
| Antifungal | A. niger | grade 0 | grade 0 | grade 0 | grade 0 |
| Activity | P. citrinum | grade 0 | grade 0 | grade 0 | grade 0 |
| | A. flavans | grade 0 | grade 0 | grade 0 | grade 0 |
| | T. mentagrophytes | grade 0 | grade 0 | grade 0 | grade 0 |
| | T. rubrum | grade 0 | grade 0 | grade 0 | grade 0 |
| | Candida albicans | grade 0 | grade 0 | grade 0 | grade 0 |

| | | | | |
|---|---|---|---|---|
| Foot-and-mouth disease Virus | not | not | not | not |
| | detected | detected | detected | detected |

From Table 6, it can be seen that the bactericidal disinfectant agents of Examples 22 to 25 were excellent in antibacterial and antifungal activities, and they were also efficient as foot-and-mouth disease inhibitors.

### EXAMPLES 26 to 28

With the compositional ratios of Table 7 below, the compositions of Examples 26 to 28 were prepared. Thereafter, their antifungal activities were determined.

TABLE 7

| | EX. 26 | EX. 27 | EX. 28 |
|---|---|---|---|
| Moutan Root Bark Extract | 15 | 10 | 5 |
| Norfloxacin | — | 5 | 2 |
| Purified Water | 25 | 20 | 25 |
| Triclosan | — | — | 1 |
| Ketoconazole | — | 5 | 2 |
| ethyl alcohol | 60 | 60 | 60 |
| A. niger | grade 0 | grade 0 | grade 0 |
| P. citrinum | grade 0 | grade 0 | grade 0 |
| A. flavans | grade 0 | grade 0 | grade 0 |
| T. mentagrophytes | grade 0 | grade 0 | grade 0 |
| T. rubrum | grade 0 | grade 0 | grade 0 |
| Candida albicans | grade 0 | grade 0 | grade 0 |

From Table 7 above, it can be seen that the compositions of Examples 26 to 28 had excellent antifungal activities. Therefore, the above compositions can be used for the treatment and prevention of skin itching, Tinea, dandruff, or athlete's foot.

### Experiment 2

The compositions of Examples 26 to 28 were charged into hand-operated sprayers. These antimicrobial agents were sprayed onto the affected parts of 20 adults whose feet were infected three times a day for 3 seconds each time, and then the treatment effects of athlete's-foot-causing pathogens and Trichophyton were investigated over the time. As the result of treatment, in the case of persons suffering from severe athlete's foot, the following symptoms were observed over the usage time.
After 1-3 days from treatment: in epithelial cells or tissues, the phenomenon of watery discharge disappeared.
After 3-5 days from treatment: cracked regions gradually healed up.
After 5-7 days from treatment: keratin was formed in dandruff region, and itching and pain disappeared.
After 7-9 days from treatment: athlete's foot seemed to be completely recovered by the formation of keratin.

In the case of persons whose athlete's feet were not severe, treatment 6 times over 3 days induced sufficient formation of keratin and thus the complete recovery of athlete's-foot-causing pathogens and Candida species, which are itch-inducing pathogens, was possible. A skin stimulation test was not separately conducted, but special lesions or side effects on the skin of the patients were not observed.

Therefore, the antimicrobial agents of Examples 26 to 28 were very effective in the prevention and treatment of athlete's foot, by causing keratinization of the tissues containing athlete's foot and thereby inducing the change in growth conditions of Trichophyton.

### Experiment 3

The antimicrobial agents of Examples 26 to 28 were treated to strains residing in skin and inducing acne and various skin diseases, and the antibacterial activities were determined. The results are as shown in Table 8 below.

TABLE 8

| | EX. 26 | EX. 27 | EX. 28 |
|---|---|---|---|
| Staphylococcus aureus | 100% | 99.9% | 99.9% |
| Staphylococcus epidermidis | 100% | 98.5% | 100% |
| Streptococcus pyogenes | 99.9% | 99.9% | 99.9% |
| Pronionbacterium acne | 99.6% | 100% | 99.6% |

From Table 8 above, it is revealed that the antimicrobial agents of Examples 26 to 28 of the present invention had excellent antibacterial activities against skin flora, and thus they can be used in cosmetics or cleansing detergents for the prevention and treatment of skin diseases.

### Experiment 4

The antimicrobial agents of Examples 26 to 28 were applied to acne-infected skin, and clinical experiments were conducted.

22 eruptive acne patients and 14 inflamed acne patients were constituted in a male to female ratio of 11:25, and they used the agents 2 to 3 times a day for 15 days. Acne treatment effects were evaluated by being observed by the naked eye. The results are exhibited in Table 9 below. As a control, EJ oriental medicine soap composition was used.

TABLE 9

| | Significant Effect | Slight Effect | No Effect | Deteriorated |
|---|---|---|---|---|
| EX. 26-28 | 9 persons | 7 persons | 2 persons | 0 person |
| Control | 0 | 7 persons | 7 persons | 4 persons |

From Table 9 above, it can be seen that the antimicrobial agents of Examples 26 to 28 of the present invention had excellent treatment effects with regard to acne, with no skin stimulation, but the control, which was used as conventional treatment agent of acne, in some cases, even deteriorated acne.

### EXAMPLES 29 to 65

### Preparation of Spray Composition for Prevention and Treatment of Athlete's Foot

250 ml of each of cyclodextrine and water were charged into a mixing container and sufficiently stirred at 750 rpm, then 75 g of natural flavor or synthetic flavor oil were slowly added thereto and stirred, and thereafter, moisture was completely eliminated therefrom and the resultant was micro-powdered to prepare the fragrance encapsulator. Also, the Moutan root bark extract used herein was a mixed extract of ethyl acetate and water, and spray compositions of Examples 29 to 65 were prepared with the compositional ratios of Table 10a and 10b below (units: wt. %).

TABLE 10a

| | | | | | Skin | |
|---|---|---|---|---|---|---|
| | Pharmacological | Fragrance | | | Permeation | |
| | Activator | Encapsulator | Solvent | Moisturizer | Enhancer | Total |
| EX. 29 | Ketoconazole 0.01 | Lavender 0.5 | Ethanol 96.49 | PEG 1.0 | Lanolin 2.0 | 100 |
| EX. 30 | Ketoconazole 0.5 | Lavender 0.5 | Ethanol 96.00 | PEG 1.0 | Lanolin 2.0 | 100 |
| EX. 31 | Ketoconazole 1.0 | Lavender 0.5 | Ethanol 95.50 | PEG 1.0 | Lanolin 2.0 | 100 |
| EX. 32 | Ketoconazole 2.0 | Lavender 0.5 | Ethanol 94.50 | PEG 1.0 | Lanolin 2.0 | 100 |
| EX. 33 | Fluconazole 0.01 | Lilac 0.5 | Ethanol 96.49 | EG 1.0 | Lanolin 2.0 | 100 |
| EX. 34 | Fluconazole 0.5 | Lilac 0.5 | Ethanol 96.00 | EG 1.0 | Lanolin 2.0 | 100 |
| EX. 35 | Fluconazole 1.0 | Lilac 0.5 | Ethanol 95.50 | EG 1.0 | Lanolin 2.0 | 100 |
| EX. 36 | Itraconazole 0.01 | Rosemary 0.5 | Ethanol 96.49 | PEG 1.0 | Lanolin 2.0 | 100 |
| EX. 37 | Itraconazole 0.5 | Rosemary 0.5 | Ethanol 96.00 | PEG 1.0 | Lanolin 2.0 | 100 |
| EX. 38 | Itraconazole 1.0 | Rosemary 0.5 | Ethanol 95.50 | PEG 1.0 | Lanolin 2.0 | 100 |
| EX. 39 | Miconazole 0.01 | peppermint 0.5 | Ethanol 96.49 | PEG 1.0 | Lanolin 2.0 | 100 |
| EX. 40 | Miconazole 0.5 | peppermint 0.5 | Ethanol 96.00 | PEG 1.0 | Lanolin 2.0 | 100 |
| EX. 41 | Miconazole 1.0 | peppermint 0.5 | Ethanol 95.50 | PEG 1.0 | Lanolin 2.0 | 100 |
| EX. 42 | Ketoconazole 1.0 | Lavender | Ethanol 70.00 | PG 1.0 | Lanolin 2.0 | 100 |
| | Fluconazole 1.0 | 0.5 | IPA 24.50 | | | |
| EX. 43 | Ketoconazole 1.0 | Lavender | Ethanol 70.00 | PG 1.0 | Lanolin 2.0 | 100 |
| | Itraconazole 1.0 | 0.5 | IPA 24.50 | | | |
| EX. 44 | Ketoconazole 1.0 | Lavender | Ethanol 70.00 | PG 1.0 | Lanolin 2.0 | 100 |
| | Terbinafine 1.0 | 0.5 | IPA 24.50 | | | |
| EX. 45 | Fenticonazole 1.0 | Musk 0.5 | IPA 95.50 | PEG 1.0 | Lanolin 2.0 | 100 |
| EX. 46 | Econazole 1.0 | Apple 0.5 | IPA 95.50 | PEG 1.0 | Lanolin 2.0 | 100 |
| EX. 47 | Bifonazole 1.0 | Lemon 0.5 | IPA 95.50 | PEG 1.0 | Lanolin 2.0 | 100 |
| EX. 48 | Oxiconazole 1.0 | Floral 0.5 | Ethanol 95.50 | EG 1.0 | Lanolin 2.0 | 100 |
| EX. 49 | Cloconazole 1.0 | Frisia 0.5 | Ethanol 95.50 | EG 1.0 | Lanolin 2.0 | 100 |
| EX. 50 | Rolcyclate 1.0 | Lavender 0.5 | Ethanol 95.50 | EG 1.0 | Lanolin 2.0 | 100 |
| EX. 51 | Amphotericin B 1.0 | Lavender 0.5 | Ethanol 95.50 | EG 1.0 | Lanolin 2.0 | 100 |
| EX. 52 | Flucytosine 1.0 | Rosemary 0.5 | IPA 95.50 | PG 1.0 | Lanolin 2.0 | 100 |
| EX. 53 | Griceofulvin 1.0 | Rosemary 0.5 | IPA 95.50 | PG 1.0 | Lanolin 2.0 | 100 |
| EX. 54 | Terbinafine 1.0 | Rosemary 0.5 | IPA 95.50 | PG 1.0 | Lanolin 2.0 | 100 |

Encapsulator: Artificially Synthetic/Natural Fragrance of Bolak (Co., Ltd.) + Cyclodextrine, IPA: isopropyl alcohol, PEG: polyethylene glycol 400, EG: ethylene glycol, PG: propylene glycol

TABLE 10b

| | Pharmacological | Fragrance | | | Skin Permeation | |
|---|---|---|---|---|---|---|
| | Activator | Encapsulator | Solvent | Moisturizer | Enhancer | Total |
| EX. 55 | Moutan Root Bark Extract 0.5 | Peppermint 0.5 | Ethanol 94.00 | PEG 2.0 | Lanolin 3.0 | 100 |
| EX. 56 | Moutan Root Bark Extract 1.0 | Peppermint 0.5 | Ethanol 93.50 | PEG 2.0 | Lanolin 3.0 | 100 |
| EX. 57 | Moutan Root Bark Extract 5.0 | Peppermint 0.5 | Ethanol 89.50 | PEG 2.0 | Lanolin 3.0 | 100 |
| EX. 58 | Moutan Root Bark Extract 10.0 | Peppermint 0.5 | Ethanol 84.50 | PEG 2.0 | Lanolin 3.0 | 100 |
| EX. 59 | Moutan Root Bark Extract 5.0 | Peppermint 0.5 | IPA 88.50 | EG 2.0 | Lanolin 3.0 | 100 |
| | Ketoconazole 1.0 | | | | | |
| EX. 60 | Moutan Root Bark Extract 5.0 | Peppermint 0.5 | IPA 88.50 | EG 2.0 | Lanolin 3.0 | 100 |
| | Fluconazole 1.0 | | | | | |
| EX. 61 | Moutan Root Bark Extract 5.0 | Peppermint 0.5 | IPA 88.50 | EG 2.0 | Lanolin 3.0 | 100 |
| | Itraconazole 1.0 | | | | | |
| EX. 62 | Moutan Root Bark Extract 5.0 | Peppermint 0.5 | IPA 88.50 | PG 2.0 | Lanolin 3.0 | 100 |
| | Miconazole 1.0 | | | | | |
| EX. 63 | Moutan Root Bark Extract 5.0 | Peppermint 0.5 | IPA 88.50 | PG 2.0 | Lanolin 3.0 | 100 |
| | Econazole 1.0 | | | | | |
| EX. 64 | Moutan Root Bark Extract 5.0 | Peppermint 0.5 | IPA 88.50 | PG 2.0 | Lanolin 3.0 | 100 |
| | Oxiconazole 1.0 | | | | | |
| EX. 65 | Moutan Root Bark Extract 5.0 | Peppermint 0.5 | IPA 88.50 | PG 2.0 | Lanolin 3.0 | 100 |
| | Terbinafine 1.0 | | | | | |

### Experiment 7

### Verification of Skin Permeation Enhancement Effect

The determination of skin permeation enhancement effects of Ketoconazole, Fluconazole, Terbinafine, and Moutan root bark was conducted using a percutaneous absorption apparatus (Franz-type diffusion cell, Hason Co., Ltd.) with regard to the skin of Guinea pigs.

With the compositional ratios of Table 11 below, compositions 1 to 8 were prepared as experimental groups, and compositions 9 and 10 were used as control groups.

TABLE 11

| | | Pharmacological | | Skin Permeation | |
|---|---|---|---|---|---|
| | | Activator | Solvent | Enhancer | Total |
| Experimental | Composition 1 | Ketoconazole 1.0 | Ethanol 97.00 | Cetyl alcohol 2.0 | 100 |
| Group | Composition 2 | Ketoconazole 1.0 | Ethanol 97.00 | TWIN 80 2.0 | 100 |
| | Composition 3 | Ketoconazole 1.0 | Ethanol 97.00 | SPAN 80 2.0 | 100 |
| | Composition 4 | Ketoconazole 1.0 | Ethanol 97.00 | Lanolin 2.0 | 100 |
| | Composition 5 | Fluconazole 1.0 | Ethanol 97.00 | Lanolin 2.0 | 100 |
| | Composition 6 | Clotrimazole 1.0 | Ethanol 97.00 | Lanolin 2.0 | 100 |
| | Composition 7 | Terbinafine 1.0 | Ethanol 97.00 | Lanolin 2.0 | 100 |
| | Composition 8 | Moutan Root Bark | Ethanol 97.0 | Lanolin 2.0 | 100 |
| | | Extract 1.0 | | | |
| Control | Composition 9 | Ketoconazole 1.0 | Ethanol 99.00 | — | 100 |
| Group | Composition 10 | Moutan Root Bark | Ethanol 99.00 | — | 100 |
| | | Extract 1.0 | | | |

1 cm² of the abdominal skin of Guinea pigs was gathered, placed in permeation cells whose diameter was 0.9 cm, and then fixed with a clamp. 0.5 ml of each of the experimental groups and control groups was applied to one side of the skin and placed at 32° C. for 24 hours. Thereafter, the permeated samples were collected from the applied skin and analyzed with HPLC to determine the degree of absorption into the skin. The results are exhibited in Table 12 below.

TABLE 12

| | | Skin Absorption Amount (μl/cm²/wt. %) | | | |
|---|---|---|---|---|---|
| | | | 8 Hours | 16 Hours | 24 Hours |
| | | 0 hour | Later | Later | Later |
| Experimental | Composition 1 | 0 | 3.11 | 7.14 | 9.44 |
| Group | Composition 2 | 0 | 6.36 | 9.55 | 11.66 |
| | Composition 3 | 0 | 7.02 | 10.88 | 12.92 |
| | Composition 4 | 0 | 10.55 | 14.82 | 21.24 |
| | Composition 5 | 0 | 9.33 | 12.69 | 19.92 |
| | Composition 6 | 0 | 9.96 | 11.23 | 19.32 |
| | Composition 7 | 0 | 10.33 | 12.22 | 20.23 |
| | Composition 8 | 0 | 4.55 | 9.65 | 15.59 |
| Control | Composition 9 | 0 | 1.42 | 3.55 | 5.95 |
| Group | Composition 10 | 0 | 2.62 | 6.78 | 8.99 |

From Table 12 above, it can be seen that the experimental groups using the skin permeation enhancers had significantly better skin permeation rate as compared with the control groups that did not use the skin permeation enhancers.

### Experiment 8

### Verification of Antibacterial and Antifungal Activity

Antibacterial and antifungal activities were determined with regard to the compositions of Examples 29 to 65.

The antibacterial test was conducted according to ASTM G22, which is the standard of the American Society for Testing and Materials, and the antifungal test was conducted according to ASTM G21.

The antibacterial and antifungal activities were observed and classified into the following grades, and the results are exhibited in Table 13 below.

(Grade)

Criteria

grade 0: No strain was grown on specimen

grade 1: Strain within 10% was grown on specimen

grade 2: Strains of about 10%˜30% were grown on specimen

grade 3: Strains of about 30%˜60% were grown on specimen

grade 4: Strains more than 60% were grown on specimen

TABLE 13

| | T. | T. | Candida | Epidermophyton | A. | P. |
|---|---|---|---|---|---|---|
| | mentagrophytes | rubrum | albicans | floccosum | niger | citrinum |
| EX. 31 | grade 0 | grade 0 | grade 0 | grade 0 | grade 0 | grade 0 |
| EX. 35 | grade 0 | grade 0 | grade 0 | grade 0 | grade 0 | grade 0 |
| EX. 38 | grade 0 | grade 0 | grade 0 | grade 0 | grade 0 | grade 0 |
| EX. 41 | grade 0 | grade 0 | grade 0 | grade 0 | grade 0 | grade 0 |
| EX. 42 | grade 0 | grade 0 | grade 0 | grade 0 | grade 0 | grade 0 |
| EX. 43 | grade 0 | grade 0 | grade 0 | grade 0 | grade 0 | grade 0 |
| EX. 44 | grade 0 | grade 0 | grade 0 | grade 0 | grade 0 | grade 0 |
| EX. 46 | grade 0 | grade 0 | grade 0 | grade 0 | grade 0 | grade 0 |
| EX. 48 | grade 0 | grade 0 | grade 0 | grade 0 | grade 0 | grade 0 |
| EX. 50 | grade 0 | grade 0 | grade 0 | grade 0 | grade 0 | grade 0 |
| EX. 52 | grade 0 | grade 0 | grade 0 | grade 0 | grade 0 | grade 0 |
| EX. 54 | grade 0 | grade 0 | grade 0 | grade 0 | grade 0 | grade 0 |
| EX. 57 | grade 0 | grade 0 | grade 0 | grade 0 | grade 0 | grade 0 |
| EX. 59 | grade 0 | grade 0 | grade 0 | grade 0 | grade 0 | grade 0 |
| EX. 61 | grade 0 | grade 0 | grade 0 | grade 0 | grade 0 | grade 0 |
| EX. 64 | grade 0 | grade 0 | grade 0 | grade 0 | grade 0 | grade 0 |
| EX. 65 | grade 0 | grade 0 | grade 0 | grade 0 | grade 0 | grade 0 |

| | E. coli | P. aeruginosa | S. typhimurium |
|---|---|---|---|
| EX. 57 | 1.5 mm ± 0.5 mm | 2.0 mm ± 0.5 mm | 2.0 mm ± 0.5 mm |
| EX. 59 | 1.0 mm ± 0.5 mm | 1.5 mm ± 0.5 mm | 1.0 mm ± 0.5 mm |
| EX. 60 | 1.5 mm ± 0.5 mm | 1.0 mm ± 0.5 mm | 1.5 mm ± 0.5 mm |
| EX. 63 | 1.0 mm ± 0.5 mm | 1.0 mm ± 0.5 mm | 1.5 mm ± 0.5 mm |
| EX. 64 | 1.0 mm ± 0.5 mm | 1.5 mm ± 0.5 mm | 1.0 mm ± 0.5 mm |

From Table 13 above, it is reveled that the antimicrobial spray compositions of the present invention had excellent antifungal and antibacterial activities against Trichophyton mentgrophytes and Trichophyton rubrum, which are athlete's-foot-causing bacteria; Candida albicans and Epidermophyton floccosum, which are skin eczema and itching-causing bacteria; A. niger, and P. citrinum. Also, the compositions of Examples 42 to 43 and 59 to 65, which used the antimicrobial substances in a mixture form, exhibited the same results.

### Experiment 9

### Verification of Antibacterial Activity against Dermatophytes of Moutan Root Bark

In order to verify the antibacterial activities of Moutan root bark extracts when they were used alone or in a mixture form, the antibacterial activities were tested with regard to the compositions of Examples 55 and 65.

The antibacterial activity was converted according to the following Equation 1, and the results are exhibited in Table 14 below.
Antibacterial Activity=((Number of Strains in Control Group−Number of Strains in Experimental Group)/Number of Strains in Control Group)×100 (Equation 1)

TABLE 14

| | | Number of Strain | | |
|---|---|---|---|---|
| | | Right After | | Reduction |
| Inoculation | | 24 Hours Later | Rate (%) | |
| S. aureus | Control | 5.0 × 10⁵ | 683 × 10⁹ | — |
| (KCTC 1916) | Group | | | |
| | EX. 55 | 5.0 × 10⁵ | 602 × 10⁸ | 91.20 |
| | EX. 57 | 5.0 × 10⁵ | 5.7 × 10³ | 99.99 |
| | EX. 58 | 5.0 × 10⁵ | 4.3 × 10² | 99.99 |
| | EX. 59 | 5.0 × 10⁵ | 4.75 × 10³ | 99.99 |
| M. luteus | Control | 5.0 × 10⁵ | 1.72 × 10¹⁰ | — |
| (KCTC 1071) | Group | | | |
| | EX. 55 | 5.0 × 10⁵ | 1.33 × 10⁹ | 92.26 |
| | EX. 57 | 5.0 × 10⁵ | 6.0 × 10² | 99.99 |
| | EX. 58 | 5.0 × 10⁵ | 3.25 × 10² | 99.99 |
| | EX. 59 | 5.0 × 10⁵ | 5.11 × 10² | 99.99 |

From Table 14 above, it can be seen that the spray composition comprising the Moutan root bark extract had bactericidal effects against skin flora. Also, Example 59, in which the Moutan root bark extract and Ketoconazole were mixed, also exhibited antibacterial activities against skin flora. Therefore, it is expected that the Moutan root bark extract can substantially prevent foul odor from a human body by exhibiting antibacterial effects against skin flora.

### Experiment 10

### Verification of Skin Stimulation

The skin stimulation degree of the antimicrobial spray compositions of the present invention was tested according to the same method as in Experiment 1.

The test was applied to 10 subjects of 19 to 34 year-old males and females of 22 years on average, and Psoriasis, Eczema, other skin lesion holders, pregnant women, nursing women, or persons who take contraceptive agents, antihistamines, etc. were excluded from this experiment. The results are exhibited in Table 15 below.

TABLE 15

| | EX. | EX. | EX. | EX. | EX. | EX. | EX. | EX. | EX. | EX. | EX. | EX. | EX. |
|---|---|---|---|---|---|---|---|---|---|---|---|---|---|
| | 31 | 35 | 38 | 41 | 57 | 58 | 59 | 60 | 61 | 62 | 63 | 64 | 65 |
| Result | — | — | — | — | — | — | — | — | — | — | — | — | — |

From Table 15 above, it can be seen that the antimicrobial spray compositions of the invention were harmless, inducing no stimulation to skin.

### EXAMPLES 66 to 82

### Preparation of Spray Composition

The spray compositions of Examples 66 to 82 were prepared with the compositional ratios of Table 16 (units: wt. %).

TABLE 16

| | Ketoconazole | Fragrance | Solvent | Moisturizer | Total |
|---|---|---|---|---|---|
| EX. 66 | 0.05 | Lavender 1 | Ethanol 98.75 | PEG 0.2 | 100 |
| EX. 67 | 0.1 | Lavender 1 | Ethanol 98.70 | PEG 0.2 | 100 |
| EX. 68 | 0.5 | Lilac 1 | Ethanol 98.3 | PEG 0.2 | 100 |
| EX. 69 | 0.7 | Lilac 1 | Ethanol 98.1 | PEG 0.2 | 100 |
| EX. 70 | 0.75 | Lilac 1 | Ethanol 98.05 | PEG 0.2 | 100 |
| EX. 71 | 1.0 | Lilac 1 | Ethanol 97.8 | PEG 0.2 | 100 |
| EX. 72 | 2.0 | Lavender 0.5 | IPA 96.80 | PEG 0.2 | 100 |
| | | Lily 0.5 | | | |
| EX. 73 | 3.0 | Lily 0.5 | IPA 96.30 | PEG 0.2 | 100 |
| EX. 74 | 4.0 | Apple 1 | IPA 94.80 | PEG 0.2 | 100 |
| EX. 75 | 2.0 | Lilac 1 | Ethanol 60.00 | PEG 0.2 | 100 |
| | | | IPA 36.80 | | |
| EX. 76 | Ketoconazole 1.90 | Lavender 1 | Ethanol 60.00 | PEG 0.2 | 100 |
| | Itraconazole 0.10 | | IPA 36.80 | | |
| EX. 77 | Fluconazole 0.05 | Lavender 1 | IPA 98.75 | PEG 0.2 | 100 |
| EX. 78 | Fluconazole 0.25 | Lavender 0.5 | IPA 98.30 | PEG 0.2 | 100 |
| | Ketoconazole 0.25 | Lily 0.5 | | | |
| EX. 79 | Fluconazole 1.0 | Lavender 0.5 | IPA 38.30 | PEG 0.2 | 100 |
| | | | Ethanol 60.00 | | |
| EX. 80 | Itraconazole 0.05 | Lavender 0.5 | Ethanol 99.15 | PEG 0.3 | 100 |
| EX. 81 | Itraconazole 0.25 | Lily 0.5 | IPA 98.70 | PEG 0.3 | 100 |
| | Ketoconazole 0.25 | | | | |
| EX. 82 | Itraconazole 1.0 | Apple 0.5 | EA 10.00 | PEG 0.3 | 100 |
| | | | IPA 88.20 | | |

### Experiment 11

### Verification of Antibacterial and Antifungal Activity

According to ASTM G21 (the standard of American Society for Testing and Materials), the spray compositions of Examples 66 to 82 were tested.

As a result, the spray compositions of Examples 66 to 82 exhibited bactericidal activities against fungi. A portion of the results is exhibited in Table 17 below.

TABLE 17

| | Antifungal Effect (Inhibition zone) | | | | |
|---|---|---|---|---|---|
| | EX. 67 | EX. 68 | EX. 69 | EX. 71 | EX. 76 |
| P. citrinum | 2 ± 0.1 mm | 6 ± 0.1 mm | 8 ± 0.1 mm | 8.5 ± 0.1 mm | 8.5 ± 0.1 mm |
| A. niger | 2 ± 0.1 mm | 9 ± 0.1 mm | 11 ± 0.1 mm | 12 ± 0.1 mm | 12 ± 0.1 mm |

From Table 17 above, it is revealed that the compositions comprising 0.1 to 1.0% by weight of Ketoconazole exhibited excellent antifungal effects against athlete's-foot-causing pathogens (Trichophyton mentgrophytes, rubrum), and skin eczema-causing pathogens (Candida albicans, Epidermophyton floccosum), and Example 76 in which Itraconazole was mixed exhibited particularly excellent antifungal effects.

### EXAMPLES 83 to 85

### Preparation of Composition for Prevention of Osmidrosis

With the compositional ratios of Table 18 below (units: wt. %), the compositions for the prevention of osmidrosis of Examples 83 to 85 were prepared.

TABLE 18

| | EX. 83 | EX. 84 | EX. 85 |
|---|---|---|---|
| Moutan Root Bark Extract | 2 | 1 | 0.5 |
| Triclosan | 0.5 | 0.5 | 0.5 |
| Magnesium Oxide (Aluminium | 4.60 | 4.60 | 4.60 |
| hydroxychloride) | | | |
| PMMA | 2.0 | 2.0 | 2.0 |
| Zeolite | 0.23 | 0.23 | 0.23 |
| DC 344 | 6.77 | 6.77 | 6.77 |
| IPM | 0.92 | 0.92 | 0.92 |
| Bisabolol | 0.02 | 0.02 | 0.02 |
| Fragrance | 0.15 | 0.15 | 0.15 |
| Liquefied Petroleum gas | 82.81 | 83.81 | 84.31 |
| Total | 100 | 100 | 100 |

### Experiment 12

1. Verification of Antibacterial Activity against osmidrosis-inducing pathogens

According to strain reduction rate determination (Shaking Flask Method), the compositions for the prevention of osmidrosis of Examples 83 to 85 were tested against Staphylococcus aureus, (KCTC1621), Stenotrophomonas maltophilia, (KCTC 2437), and Candida albicans, (KCTC 7729), which are typical Osmidrosis-inducing pathogens, and the results are exhibited in Table 19.

TABLE 19

| | EX. 83 | EX. 84 | EX. 85 |
|---|---|---|---|
| S. aureus, KCTC1621 | 99.99% | 99.99% | 99.99% |
| S. maltophilia, KCTC 2437 | 99.99% | 99.99% | 99.99% |
| C. albicans, KCTC 7729 | 99.99% | 99.99% | 99.99% |

From Table 19, it can be seen that the compositions of Examples 83 to 85 had superior bactericidal effects against osmidrosis —inducing pathogens.

2. Comparison of Deodorization Effect against Osmidrosis and Foul Odor.

The test was conducted with regard to 30 females and 30 males of 20˜45 years who perceived unpleasant osmidrosis from themselves and believed that the osmidrosis become a source of annoyance to other people. The compositions of Examples 83 to 85 were sprayed onto them three times (morning, afternoon, evening) a day for 4 weeks, the deodorizing effects against osmidrosis were evaluated by panels according to the criteria as shown in Table 20 below, and their averages are exhibited in Table 21.

Furthermore, to evaluate skin stimulation, skin stimulation was tested according to the same method as in Experiment 1, and the results are exhibited in Table 22.

TABLE 20

| Criteria | Contents |
|---|---|
| 4 | Deodorizing effects are excellent. |
| 3 | Deodorizing effects seem to exist, |
| | but they are not sure. |
| 2 | Slight deodorizing effects seem to exist, |
| | but they are not sure. |
| 1 | There are neither deodorizing effects nor |
| | deterioration of osmidrosis. |
| 0 | There are no deodorizing effects and rather, |
| | osmidrosis is deteriorated. |

TABLE 21

| | Score (evaluated | Score (evaluated |
|---|---|---|
| | by panels | by sensory evaluation |
| | themselves) | experts) |
| EX. 84 | 3.8 | 3.9 |
| EX. 85 | 3.8 | 3.7 |
| EX. 86 | 3.7 | 3.7 |

TABLE 22

| | Skin Stimulation |
|---|---|
| EX. 84 | — |
| EX. 85 | — |
| EX. 86 | — |

From Tables 21 and 22, it can be seen that when the compositions for the prevention of osmidrosis of the present invention were used in the form of powder spray aerosols, they exhibited excellent deodorizing effects against unpleasant osmidrosis and foul odor, and induced no skin stimulation.

### EXAMPLES 86 to 88

### Composition for Oral Cleansing

With the compositional ratios of Table 23 below (units: wt. %), the compositions for oral cleansing were prepared.

TABLE 23

| | EX. 86 | EX. 87 | EX. 88 |
|---|---|---|---|
| Moutan Root Bark Extract | 1 | — | 0.5 |
| Xylitol | — | 1 | 0.5 |
| Glycerin | 8 | 8 | 8 |
| Sodium Fluoride | 0.02 | 0.02 | 0.02 |
| Sodium Saccharin | 0.5 | 0.5 | 0.5 |
| Ethanol | 12 | 12 | 12 |
| Polydimethylsiloxan | 20 | 20 | 20 |
| Emulsion | 0.04 | 0.04 | 0.04 |
| Purified Water | up to 100 | up to 100 | up to 100 |
| Total | 100 | 100 | 100 |

1. Antibacterial Test against Oral Microorganism

According to the strain reduction rate method (Shaking Flask Method), antibacterial test was conducted. As strains, Streptococcus mutans (KCTC 3065) and Streptococcus mitis (KCTC 3556) were used, and the results are exhibited in Table 24 below.

TABLE 24

| | EX. 86 | EX. 87 | EX. 88 |
|---|---|---|---|
| Streptococcus mitis | 98.56% | 85.45% | 99.9% |
| Streptococcus mutans | 97.77% | 80.44% | 100% |

2. Verification of Durability of Antibacterial Effect

The compositions of Examples 86 to 88 were added to Streptococcus mutans, and strain reduction rate was measured over time. The results are exhibited in Table 25 below.

TABLE 25

| | 6 hr | 12 hr | 24 hr | 48 hr | 72 hr |
|---|---|---|---|---|---|
| EX. 86 | 97.55% | 97.70% | 97.77% | 93.25% | 80.44% |
| EX. 87 | 83.23% | 81.15% | 80.44% | 60.35% | 43.25% |
| EX. 88 | 99.99% | 100% | 100% | 100% | 98.33% |

3. Verification of Pain Relief Effect

After the compositions of Examples 86 to 88 were applied, the number of subjects who did not feel pain any more was counted, and the results are exhibited in Table 26 below.

TABLE 26

| | | | | | | | Longer |
|---|---|---|---|---|---|---|---|
| | 1 | 2 | 3 | 4 | 5 | 6 | Than 1 |
| | Day | Day | Day | Day | Day | Day | Week |
| EX. 86 | 2 | 2 | 4 | 3 | 3 | 0 | 6 |
| EX. 87 | 3 | 1 | 2 | 4 | 2 | 1 | 7 |
| EX. 88 | 2 | 4 | 6 | 3 | 2 | 2 | 1 |

4. Verification of Halitosis Inhibition Effect

It was tested whether the elimination efficacy of halitosis actually occurs when the compositions for oral cleansing of Examples 86 to 88 are applied to humans.

The halitosis-detecting component contained in garlic was defined to methyl mercaptan, and its elimination rate was measured in terms of deodorization rate. For the test, a halitosis-detector was used (Dr. Etiquette DE-160 (Winners Japan Co., Ltd.), which is a device to measure the amount of methyl mercaptan, and five people was tested.

The subjects were forced not to ingest anything within 2 hours of the test, and they chewed 0.5 g of garlic for 2 minutes and then sprayed their mouths with the compositions of Examples 86 to 88 for 1 minute. Thereafter, the concentration of methyl mercaptan within each mouth was measured six times at ten minute intervals. Also, as a control, subjects chewed 0.5 g of garlic for 2 minutes, and then the concentration of methyl mercaptan within their mouth was measured six times at ten minute intervals.

The deodorization rate was calculated according to the following Equation 2.
Deodorization Rate (%)=((S−H)/S)×100 (Equation 2)
S: Conc. of Methyl Mercaptan Right After Application of Composition for Oral Cleansing (ppm)
H: Conc. of Methyl Mercaptan Measured at Ten Minutes' Interval After Application of Composition for Oral Cleansing (ppm)

The deodorization rate results, as shown in Table 27, show that the compositions for oral cleansing of the present invention had durable inhibition effects against halitosis.

TABLE 27

| | 10 hr | 20 hr | 30 hr | 40 hr | 50 hr | 60 hr |
|---|---|---|---|---|---|---|
| Control(No | 10.50 | 15.37 | 18.77 | 34.55 | 36.53 | 42.45 |
| Treatment) | | | | | | |
| EX. 86 | 75.44 | 77.55 | 79.32 | 83.56 | 81.45 | 81.25 |
| EX. 87 | 73.56 | 74.77 | 79.45 | 80.22 | 81.56 | 79.66 |
| EX. 88 | 88.55 | 89.78 | 91.45 | 92.11 | 93.45 | 93.78 |

As revealed in the above, the compositions for oral cleansing of Examples 86 to 88 had excellent durable deodorization effects against halitosis, and Example 88, in which the Moutan root bark extract and the compounds were used together, showed particularly excellent results.

### EXAMPLES 89 to 91

### Detergent Composition Containing Moutan Root Bark Extract

With the compositional ratios of Table 28 below (units: wt. %), the detergent compositions were prepared.

TABLE 28

| | EX. 89 | EX. 90 | EX. 91 |
|---|---|---|---|
| Moutan Root Bark Extract | 0.5 | — | 0.5 |
| Triclosan | — | 0.5 | 0.5 |
| Alkyl Benzene Sulfonic Acid | 11.0 | 11.0 | 11.0 |
| Magnesium Oxide | 0.69 | 0.69 | 0.69 |
| Alkylether Sulfate Sodium Salt (27%) | 15.0 | 15.0 | 15.0 |
| Ethyl Alcohol | 0.5 | 0.5 | 0.5 |
| Coconut Fatty Acid Diethanolamide | 2.0 | 2.0 | 2.0 |
| Triethanolamine | 0.5 | 0.5 | 0.5 |
| Hydroxy Ethylcellulose | 0.05 | 0.05 | 0.05 |
| Water | up to 100 | up to 100 | up to 100 |
| Total | 100 | 100 | 100 |

1. Evaluation of Bactericidal Ability of Dish Towel

To a solution prepared by mixing E. coli(KCTC 1682), S. aureus (KCTC 1621), and P. aeruginosa (KCTC 2004) and diluting them (2.0×10⁶/ml) were added pieces of test cotton (white cotton pieces sterilized at 121° C. for 15 minutes) to thereby contaminate them, and they were then washed with the compositions of Examples 89 to 91 for 1 minute and mounted on media for bacterial culture and left at 37° C. Thereafter, the proliferation degree of bacteria was evaluated according to the following criteria, and the results are exhibited in Table 29 below.

Bacteria Proliferation Degree
⊚: Completely Sterilized, 0: Sterilized, Δ: Inhibited, x: Proliferated

2. Evaluation of Bactericidal Ability

With regard to Salomella typhimurium (KCTC1925) and Shigella flexneri (KCTC 2008), the same experiment as above was conducted. The bacteria culture was carried out at 40° C. for 24 hours.

3. Evaluation of Deodorization Ability

The compositions of Examples 89 to 91 were charged into the sealed containers and equal amounts of foul odor sources (3 kinds), were respectively added thereto, and after a predetermined time the remaining portion of foul odor that was not deodorized was absorbed to detector tubes to thereby determine the concentration of the remaining gas (Use of Gastec Detector Tubes).

Source of Foul Odor
Ammonia: Use of Ammonia Detector Tube, Use of 0.03% Aqueous Solution, 0.5 ml
Amine: Use of Amines Detector Tube, Use of 0.3% aqueous solution, 0.5 ml
Mercaptan: Use of Mercaptan Detector Tubes, Use of 0.1% Benzene Solution, 0.1 ml.

Evaluation (Evaluated by the Detected Concentration in the Detector Tubes)
More than 80 ppm: No Deodorization Ability (x)
50-80 ppm: Slight Deodorization Ability (Δ)
20-50 ppm: Presence of Deodorization Ability (O)
Less than 20 ppm: Excellent Deodorization Ability (©).

The following Table 29 exhibits bactericidal and deodorizing ability of the compositions of Examples 89 to 91.

TABLE 29

| | Dish Towel | | | Deodorizing Ability | | |
|---|---|---|---|---|---|---|
| | Bactericidal | Bactericidal Ability | | | Methyl | |
| | Ability | S. typhimurium | S. flexneri | Ammonia | Mercaptan | Trimethylamine |
| EX. 89 | ◯ | ⊚ | ◯ | ◯ | Δ | ◯ |
| EX. 90 | ⊚ | ⊚ | ⊚ | Δ | Δ | Δ |
| EX. 91 | ⊚ | ⊚ | ⊚ | ⊚ | ◯ | ⊚ |

### EXAMPLES 92 to 94

### Soap Composition

With the compositional ratios of Table 30 below (units: wt. %), soap compositions were prepared.

TABLE 30

| | EX. 92 | EX. 93 | EX. 94 |
|---|---|---|---|
| Moutan Root Bark Extract | 2 | — | 2 |
| Ketoconazole | — | 2 | 1 |
| Purified Water | 2.0 | 2.0 | 2.0 |
| Triclosan | — | 1 | 1 |
| Mineral Oil | 1.0 | 1.0 | 1.0 |
| Polyethylene glycol | 0.5 | 0.5 | 0.5 |
| Fragrance | 1.2 | 1.2 | 1.2 |
| Tribromsalane | 0.1 | 0.1 | 0.1 |
| Hexachlorofen | 0.1 | 0.1 | 0.1 |
| Ethylenediamineacetate | 0.1 | 0.1 | 0.1 |
| Titanium Dioxide | 0.1 | 0.1 | 0.1 |
| Soap Base | up to 100 | up to 100 | up to 100 |
| Total | 100 | 100 | 100 |

1. Antibacterial Activity

The antibacterial activities of the compositions of Examples 92 to 94 were investigated through the formation of inhibition zones, and the results are exhibited in Table 31 below.

TABLE 31

| | EX. 92 | EX. 93 | EX. 94 |
|---|---|---|---|
| B. cereus | 3 | 10 | 10 |
| B. subtilis | 2 | 12 | 12 |
| E. coli | 3 | 0 | 3 |
| K. pnewmoniae | 3 | 0 | 3 |
| S. aureus | 4 | 12 | 12 |
| S. typhymurium | 5 | 0 | 5 |
| A. niger | grade 2 | 3 | 3 |
| A. flavans | grade 1 | 4 | 4 |
| P. citrinum | grade 2 | 2 | 2 |
| T. mentagrophytes | 3 | 15 | 15 |
| T. rubrum | 3 | 12 | 12 |

From Table 31 above, it can be seen that in cases comprising both the Moutan root bark extract and the compounds, they exhibited broad antibacterial effects throughout gram positive bacteria, gram negative bacteria, and fungi.

2. Deodorization Effect

The deodorization effects of the compositions of Examples 92 to 94 were measured, and the results are exhibited in Table 32.

TABLE 32

| | Deodorizing Ability | | |
|---|---|---|---|
| | Ammonia | Methyl Mercaptan | Trimethylamine |
| EX. 92 | ◯ | Δ | ◯ |
| EX. 93 | Δ | Δ | Δ |
| EX. 94 | ⊚ | ◯ | ⊚ |

As described above, the Moutan root bark extract of the present invention has excellent antimicrobial activity. Therefore, the Moutan root bark extract can be applied to medicines, food additives, cosmetics, bactericidal/disinfectant agents, and detergents to effectively prevent foot-and-mouth disease viruses, skin-itching, Tinea, dandruff, osmidrosis, halitosis, decayed teeth, or athlete's feet.

1. A composition for the prevention and treatment of athlete's foot, comprising a Moutan root bark extract as an active ingredient.

2. The composition for the prevention and treatment of athlete's foot of claim 1, wherein said Moutan root bark is prepared by extracting the Moutan root bark with one or more solvents selected from the group consisting of water, ethyl acetate butyl acetate, ethyl alcohol, isopropyl alcohol, butyl alcohol, hexane, chloroform, ethylene glycol, propylene glycol, propanol, acetone, benzene, ethanol, methanol, and butanol.

3. The composition for the prevention and treatment of athlete's foot of claim 1, further comprising one or more compounds selected from the group consisting of Ketoconazole, Itraconazole, Fluconazole, Miconazole, Clotrimazole, Fenticonazole, Econazole, Bifonazole, Oxiconazole, Cloconazole, Rolcyclate, Amphotericin B, Flucytosine, Griceofulvin, Terbinafine, Nystatin, Tolnaftate, Naftifine, Haloprogin, Ciclopirox, and Triclosan.

4. The composition for the prevention and treatment of athlete's foot of claim 1, wherein said composition for the prevention and treatment of athlete's foot is applied in the form of sprays.

5. The composition for the prevention and treatment of athlete's foot of claim 1, wherein said composition for the prevention and treatment of athlete's foot is a medicine, a cleansing agent, or a cosmetic.

6. A composition for the prevention of osmidrosis caused by Stenotrophomonas maltophilia, comprising a Moutan root bark extract as an active ingredient.

7. The composition for the prevention of osmidrosis of claim 6, wherein said Moutan root bark extract is prepared by extracting the Moutan root bark with one or more solvents selected from the group consisting of water, ethyl acetate butyl acetate, ethyl alcohol, isopropyl alcohol, butyl alcohol, hexane, chloroform, ethylene glycol, propylene glycol, propanol, acetone, benzene, ethanol, methanol, and butanol.

8. The composition for the prevention of osmidrosis of claim 6, wherein said composition for the prevention of osmidrosis is applied in the form of sprays, liquids and solutions, sticks, gels, creams, and cleansing agents.

9. An anti-acne composition comprising a Moutan root bark extract as an active ingredient, wherein the anti-acne composition has an anti-microbial activity for Pronionbacterium acne.

10. The anti-acne composition of claim 9, wherein said Moutan root bark extract is prepared by extracting the Moutan root bark with one or more solvents selected from the group consisting of water, ethyl acetate butyl acetate, ethyl alcohol, isopropyl alcohol, butyl alcohol, hexane, chloroform, ethylene glycol, propylene glycol, propanol, acetone, benzene, ethanol, methanol, and butanol.

11. The anti-acne composition of claim 9 or 10, characterized in that said anti-acne composition is a medicine, a cleaning agent, or a cosmetic.

12. An anti-microbial composition in the form of liquids and solutions, gels, solids, aerosols, or sprays comprising a Moutan root bark extract; and one or more compounds selected from the group consisting of Ketoconazole, Itraconazole, Fluconazole, Miconazole, Clotrimazole, Fenticonazole, Econazole, Bifonazole, Oxiconazole, Cloconazole, Rolcyclate, Amphotericin B, Flucytosine, Griceofulvin, Terbinafine, Nystatin, Tolnaftate, Naftifine, Haloprogin, Enosaxin, Norfloxacin, Cifloxacin, Acyclovir, Ribavirin, Triclosan, and Ciclopirox
and wherein the anti-microbial composition has an anti-microbial for E. coli 0-157:H-7, Streptococcus pyogenes, Salmonella choleraesuis, Pasteurella haemolytica, Listeria monocytogens, Candida albicans, A. niger, Aspergillus niger, Penillium citrinum, Aspergillus falvus, Epidermophyton Floccosum, Pseudomonas aeruginosa, Trichophyton mentagrophytes, Trichophyton rubrum, Pronionbacterium acne, Stenotrophomonas maltophilia, Streptococcus mitis and Streptococcus mutans.

13. The anti-microbial composition of claim 12, wherein said anti-microbial composition comprises said Moutan root bark extract and the compounds in a ratio of 1:5 to 5:1 by weight.

14. The anti-microbial composition of claim 12, further comprising a skin moisturizer, a skin permeation enhancer, a fragrance, a fragrance encapsulation carrier, an organic solvent, or a filler.

15. The anti-microbial composition of claim 14 comprising said compounds in an amount of 0.001 to 20%; said skin moisturizer in an amount of 0.05 to 5%; said skin permeation enhancer in an amount of 0.1 to 10%; said fragrance in an amount of 0.05 to 2% by weight; said fragrance encapsulation carrier in an amount of 0.1 to 10% by weight; said filler in an amount of 0.01 to 50% by weight; and said organic solvent in the remaining amount.

16. The anti-microbial composition of claim 14, wherein said skin permeation enhancer is one or more compounds selected from the group consisting of polyethylene glycol monolaurate (pegml), glycerol monolaurate, propylene glycol monolaurate, eucalyptol, lecithin, 1-substituted azacycloheptane-2-one, 1-n-dodecyl cycleazacyclohepta-2-one, cetyl alcohol, stearyl alcohol, myrist alcohol, polyethylene sorbitan fatty acid ester, dodecyl amine, and lanolin.

17. The anti-microbial composition of claim 14, wherein said skin moisturizer is one or more compounds selected from the group consisting of ethylene glycol, propylene glycol, butylen glycol, hexylen glycol, polyethylene glycol (PEG) 200 to 600, polypropylene glycol (PPG), glycol ester and ether, alkyl ester of polyethylene glycol, alkyl ester of polypropylene glycol, carboxylic ester of polyethylene glycol, carboxylic ester of polypropylene glycol, sorbitol, trihydroxy stearine, and polyhydric alcohol derivatives.

18. The anti-microbial composition of claim 14, wherein said fragrance is one or more fragrances selected from the group consisting of lavender, lemon, floral, herb, apple, strawberry, lily, frisia, lilac, peppermint, rosemary, freshmint, spearmint, olive, kiwi, rose, acacia, pheromone, Chinese quince and pine flavors.

19. The anti-microbial composition of claim 14, wherein said fragrance encapsulation carrier is dextrine or cyclodextrine.

20. The anti-microbial composition of claim 14, wherein said organic solvent is selected from the group consisting of ethanol, isopropanol, and a mixture thereof.

21. The anti-microbial composition of claim 14 wherein said filler is a liquefied propane gas.

22. The anti-microbial composition of claim 12, wherein said anti-microbial composition is a detergent composition, a composition for controlling dandruff-causing pathogens and odor, a bactericidal/disinfectant composition, a composition for the sterilization and disinfection of animals and animal breeding farms, or an anti-foot-and-mouth disease composition.

23. A composition for oral cleansing comprising
a Moutan root bark extract; and
one or more compounds selected from the group consisting of xylitol, propolis, triclosan, chlorohexidine gluconate (XII), cetyl pyridinium chloride (XIII), isopropylmethylphenol, hitokitiol, glytylitylic acid, and allantoin
and wherein the composition has an anti-microbial activity for Streptococcus mitis and Streptococcus mutans.
